(19) United States
(12) Patent Application Publication
(10) Pub. No.: US 2020/0129564 A1
(43) Pub. Date: Apr. 30, 2020

(54) COMPOSITIONS AND METHODS FOR TREATING WOUNDS

(71) Applicant: PROMEND ANIMAL HEALTH, INC., GERMANTOWN, TN (US)

(72) Inventors: SCOTT P. NOEL, GERMANTOWN, TN (US); JOHN KIRK SHUMPERT, GERMANTOWN, TN (US); ALEX GREENE, GERMANTOWN, TN (US); WILLIAM BRIAN AUSTIN, GERMANTOWN, TN (US)

(73) Assignee: PROMEND ANIMAL HEALTH, INC., GERMANTOWN, TN (US)

(21) Appl. No.: 16/619,778

(22) PCT Filed: May 30, 2018
(86) PCT No.: PCT/US2018/035093

§ 371 Date: Dec. 5, 2019

Related U.S. Application Data
(60) Provisional application No. 62/515,896, filed on Jun. 6, 2017.

(51) Int. Cl. A61K 35/644; A61K 31/722; A61K 31/19; A61K 31/155; A61K 33/38; A61K 31/496; A61K 33/20; A61K 9/00; A61P 17/02; A61P 31/04; A61K 9/06

(52) U.S. Cl. CPC A61K 35/644; A61K 31/722; A61K 31/19; A61K 31/155; A61K 33/38; A61K 45/06; A61K 33/20; A61K 9/0014; A61P 17/02; A61P 31/04; A61K 9/06; A61K 31/496

(57) ABSTRACT

The present invention provides compositions and methods that promote wound healing. The invention provides a composition comprising an effective amount of a biopolymer, an antiseptic, an anti-inflammatory agent, and other agents.

### CROSS-REFERENCE TO RELATED APPLICATION

This application claims the benefit of and priority to U.S. Provisional Patent Application Ser. No. 62/515,896, filed Jun. 6, 2017, which is incorporated herein by reference in its entirety.

### BACKGROUND OF THE INVENTION

Breaks to the skin can occur due to a variety of insults, including trauma. Wound healing is a complex physiological process that can be delayed due to contamination with a variety of pathogens, including bacteria, fungi, and other microbes. Wound dressings that promote moisture and that reduce or prevent infection create a protected healing environment that provides for optimal healing.

### SUMMARY OF THE INVENTION

As described below, the present invention provides compositions and methods that promote wound healing.

In one aspect, the invention provides a composition comprising an effective amount of a biopolymer, an antiseptic, and an anti-inflammatory agent. In one embodiment, the composition further contains any one or a combination of acetic, propionic, citric, lactic, hypochlorous, and phosphoric acid. In another embodiment, the biopolymer is any one or a combination of chitosan, chitin, collagen, alginate, or dextran. In another embodiment, the composition further contains a natural healing agent and/or a bittering agent in an amount sufficient to prevent licking or ingestion of the composition. In another embodiment, the natural healing agent is honey.

In another aspect, the invention provides a composition comprising an effective amount of a biopolymer selected from the group consisting of chitosan, collagen, cellulose, alginate, and dextran; an antiseptic selected from the group consisting of chlorhexidine gluconate, povidone iodine, alcohol, benzalkonium chloride, benzethonium chloride, and parachlorometaxylenol (PCMX); and an acid that is acetic acid or citric acid

In another aspect, the invention provides a composition comprising an effective amount of chitosan, acetic acid, and chlorhexidine digluconate in an excipient for topical administration.

In another aspect, the invention provides an ointment, spray, gel, or hydrogel composition comprising an effective amount of chitosan, acetic acid, and chlorhexidine digluconate in an excipient for topical administration. In various embodiments of the above-aspects, the composition further contains silver nanoparticles. In various embodiments of the above-aspects, the composition is a liquid, gel, paste, semi-solid, or solid. In various embodiments of the above-aspects, the composition is a solid or semi-solid wound dressing or bandage. In other embodiments of the above-aspects, the composition comprises between about 1-10% (e.g., 1, 2, 3, 4, 5, 6, 7, 8, 9, 10%) chitosan. In other embodiments of the above-aspects, the composition comprises between about 1-3% (e.g., 1, 2, 3%) acetic acid. In other embodiments of the above-aspects, the composition comprises between about 0.01-3% (0.01, 0.02, 0.05, 0.1, 0.2, 0.5, 1.0, 2.0, 3.0%) chlorhexidine digluconate. In other embodiments of the above-aspects, the composition comprises electrolized water containing dissolved sodium chloride. In other embodiments of the above-aspects, the composition comprises hypochlorous acid or sodium hydroxide. In other embodiments of the above-aspects, the composition comprises ketoconazole. In other embodiments of the above-aspects, the composition comprises sodium hypochlorite (NaOCl). In other embodiments of the above-aspects, the composition comprises deacetylated chitosan. In other embodiments of the above-aspects, the degree of deacetyalation is between 51-99%. In other embodiments of the above-aspects, the composition is characterized by a viscosity greater than 500 centipoise (cP). In other embodiments of the above-aspects, the composition further contains a fruit, vegetable, or plant-based pomace, powder or liquid derived from a berry, apple, citrus fruit, beet, root, or banana.

In yet another aspect, the invention provides a method for inhibiting the proliferation of Gram-negative or Gram-positive bacteria, the method comprising contacting the bacteria (e.g., methicillin-resistant Staphylococcus aureus (MRSA) or Pseudomonas aeruginosa) with a composition of any previous aspect or otherwise described herein.

In yet another aspect, the invention provides a method for treating a wound in a mammal the method comprising contacting the wound with a composition comprising a composition of any previous aspect or otherwise described herein. In one embodiment, the mammal is a human, equine, canine, or feline. In another embodiment, the method treats a topical, superficial or soft tissue infection. In another embodiment, the method treats an umbilical cord or navel and prevents infection, bacterial contamination, or aids in umbilical drying out. In another embodiment, the method the method treats or prevents itching, irritated skin, and hot spots.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound.

By “alginate” is meant the sodium salt of alginic acid. In particular embodiments, alginic acid refers to a linear copolymer with homopolymeric blocks of (1-4)-linked 1-D-mannuronate (M) and its C-5 epimer.alpha.-L-guluronate (G) residues, respectively, covalently linked together in different sequences or blocks.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antimicrobial” is meant an agent that inhibits or stabilizes the proliferation or survival of a microbe. In one embodiment, a bacteriostatic agent is an antimicrobial. In other embodiments, any agent that kills a microbe (e.g., bacterium, fungus, virus) is an antimicrobial.

By “anti-inflammatory” is meant an agent that reduces the severity or symptoms of an inflammatory reaction in a tissue.

By “cell migration” is meant the movement of a cell in, over, or through a substrate. Cell migration is typically measured in a cell migration or wound healing assay as described herein.

By “chitosan” is meant a chitin-derived polymer that is at least 20% deacetylated. Preferably, chitosan is at least about 50% deacetylated. Chitin is a linear polysaccharide consisting of (1-4)-linked 2-acetamido-2-deoxy-b-D-glucopyranose. Chitosan is a linear polysaccharide consisting of (1-4)-linked 2-amino-2-deoxy-b-D-glucopyranose.

By “acid treated chitosan” is meant chitosan that is solubilized in an acidic solution.

By “collagen” is meant a protein component of an extracellular matrix having a tertiary structure that includes polypeptide chains intertwining to form a collagen triple helix or having a characteristic amino acid composition comprising Gly-X-Y repeat units, or a fragment thereof. Collagens useful in the methods of the invention include any collagen known in the art (e.g., one of collagen type 1-29).

By “compound” is meant any small molecule chemical compound, antibody, nucleic acid molecule, or polypeptide, or fragments thereof.

By “clinician” is meant any healthcare provider. Exemplary clinicians include, but are not limited to, doctors, veterinarians, osteopaths, physician's assistants, emergency medical technicians, medics, nurse practitioners, and nurses.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “effective amount” is meant the amount of an agent required to ameliorate the symptoms of a disease relative to an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “effective amount” is meant the amount of an agent described herein required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, “obtaining” as in “obtaining an agent” includes synthesizing, purchasing, or otherwise acquiring the agent.

By “polymer” is meant a natural or synthetic organic molecule formed by combining smaller molecules in a regular pattern.

By “reference” is meant a standard or control condition.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

By “wound” is meant any damage to the skin, epidermis or connective tissue whether by injury or by disease and as such is taken to include, but not to be limited to, cuts, punctures, surgical incisions, ulcers, pressure sores, burns, including burns caused by heat, freezing, chemicals, electricity and radiation, dermal abrasion or assault, osteomyelitis and orthopaedic wounds. The wound may be infected. Additionally, the wound may be chronic or acute.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A provides an image showing zone of inhibition (ZOI) results tested against Methicillin-resistant Staphylococcus aureus (MRSA).

FIG. 1B provides an image of a turbidity plate with various veterinary products tested for their ability to inhibit MRSA.

FIG. 1C provides a graphical representation of ZOI measured around discs with loaded solutions.

FIG. 1D provides a scatter plot showing percentage of viable bacteria in contrast to saline controls

FIG. 2A is a scatter plot showing FIG. 5 viability percentage for 6 hours of direct contact with pre-formed biofilm, (*) significant difference between groups and PBS (p<0.05).

FIG. 2B is a scatterplot showing viability percentage for 24 hours of direct contact with pre-formed biofilm, (*) significant difference between groups and PBS (p<0.05).

FIGS. 3A and 3B show photographs before and after treatment of a canine with demodectic mange. Treatment was carried out using a spray formulation described herein containing chitosan and chlorhexidine.

FIGS. 4A and 4B show photographs before and after treatment of a horse with a degloved hoof injury. Treatment was carried out using a spray formulation described herein containing chitosan and chlorhexidine.

FIGS. 5A and 5B show photographs before and after treatment of a horse with a puncture wound to the chest caused by running into a broken gate. Treatment was carried out using a spray formulation described herein containing chitosan and chlorhexidine.

FIGS. 6A and 6B show photographs before and after treatment of a horse with a foreleg wound caused by running into a barbed wire fence. Treatment was carried out using a spray formulation described herein containing chitosan and chlorhexidine.

### DETAILED DESCRIPTION OF THE INVENTION

The invention provides compositions comprising a biopolymer (chitosan, chitin, collagen, cellulose, alginates, honey, dextran), an acidic solvent (e.g., acetic acid, citric acid), and an antiseptic (e.g., chlorhexidine), and in some embodiments a nanoparticle (e.g., silver, magnesium) for use in treating wounds. In particular embodiments, the compositions of the invention feature natural healing agents including but not limited to manuka honey.

The invention is based, at least in part, on the discovery that compositions comprising chitosan, chlorhexidine, and silver are particularly useful for inhibiting the proliferation of Gram-positive and Gram-negative bacteria, and for healing wounds in vivo. In addition, the invention provides wound healing compositions (e.g., liquid, gel, paste, semi-solids) and structures for use as a dressing to retain moisture within the wound, to protect the wound and wound environment from contamination, and to inhibit infection.

Infections of traumatic wounds or post-surgical wounds pose a threat to animal health and present challenges for veterinarians. The growing incidence of drug-resistant infections is particularly concerning. Methicillin-resistant Staphylococcus aureus (MRSA) can be carried by horses and humans and can cause infectious sequelae in horses, such as skin and soft tissue MRSA infections, bacteraemia, septic arthritis, osteomyelitis, and implant-related infections. Common preventative measures include hygienic routines, wound debridement and irrigation, antibiotic therapy, and wound care sprays. The use of certain prophylactic antibiotics, such as gentamicin, has been found to increase the risk of multidrug resistant infection. Bacterial attachment, or biofilm, to tissue or implant materials can increase the pathogenicity of infectious bacteria and can be particularly difficult to treat.

Many commercially available veterinary wound care sprays contain active ingredients that inhibit bacterial growth. The in vitro activity of commercially available products against MRSA growth and biofilm was tested relative to compositions of the invention. Compositions of the invention comprising chlorhexidine and silver were surprisingly effective for inhibiting the growth and proliferation of Gram-positive and Gram-negative bacteria. Compositions of the invention comprising chitosan, chlorhexidine and silver were effective in promoting wound healing in vivo.

### Chitosan

Chitosan is a naturally occurring linear polysaccharide composed of randomly distributed ß-(1-4)-2-amino-2-D-glucosamine (deacetylated) and ß-(1-4)-2-acetamido-2-D-glucoseamine (acetylated) units. Chitosan is derived from chitin, a naturally occurring polymer. Chitin is a white, hard, inelastic, nitrogenous polysaccharide isolated from fungi, mollusks, or from the exoskeletons of arthropods (e.g., crustaceans, insects). The major procedure for obtaining chitosan is the alkaline deacetylation of chitin with strong alkaline solution. Generally, the raw material is crushed, washed with water or detergent, and ground into small pieces. After grinding, the raw material is treated with alkali and acid to isolate the polymer from the raw crushed material. The polymer is then deacetylated by treatment with alkali. Chitin and chitosan differ in their degrees of deacetylation (DDA). Chitin has a degree of deacetylation of 0% while pure chitosan has a degree of deacetylation of 100%. Typically, when the degree of deacetylation is greater than about 50% the polymer is referred to as chitosan.

Chitosan is a cationic weak base that is substantially insoluble in water and organic solvents. Typically, chitosan is fairly soluble in dilute acid solutions, such as acetic, citric, oxalic, propionic, ascorbic, hydrochloric, formic, and lactic acids, as well as other organic and inorganic acids. Chitosan's charge gives it bioadhesive properties that allow it to bind to negatively charged surfaces, such as biological tissues present in a gastrointestinal tract of an animal.

In the body chitosan is degraded by lysozyme, N-acetyl-o-glucosaminidase and lipases. Lysozyme degrades chitosan by cleaving the glycosidic bonds between the repeating chitosan units. The byproducts of chitosan degradation are saccharides and glucosamines that are gradually absorbed by the body. This biopolymer material has been used medically, and is valued for its biocompatibility, degradation and absorption properties, hemostatic properties, and for promoting the healing process in damaged tissues. Chitosan has also been linked in scientific literature as being antimicrobial, bacteriostatic, anti-inflammatory, and for reducing itching. Chitosan has been used as coating, a composition binder, and as an active ingredient in pharmaceutical applications.

### Collagen

Collagen is the most abundant structural protein in the body, existing as the foremost component of the extracellular matrix (ECM). Most types of collagen contain a unique tertiary structure that includes three individual right-handed helical polypeptide chains intertwining to form a left-handed helix. Collagen has a characteristic amino acid composition comprised of Gly-X-Y repeat units. Collagen is used in a variety of medical applications including hemostatic materials, biocompatible coatings, drug delivery and tissue engineering. Collagen-based biomaterials are also used in soft-tissue engineering and repair. In the past two decades, a multitude of medical products composed of collagen have been approved by the FDA, and many are available as commercial products, including collagen-based corneal shields, anti-infectious catheters, tissue sealants, hemostatic sponges, and topical wound dressing products. Collagen is also used as a tissue engineering substrate for skin, bone, and blood vessel replacement.

### Antiseptics

In particular embodiments, a composition of the invention includes an antiseptic (e.g., chlorhexidine). Chlorhexidine is active against Gram-positive and Gram-negative organisms, aerobes, anaerobes, and yeasts. In low concentrations it remains effective without causing toxicity or impairing healing. Other antiseptics useful in compositions of the invention include, but are not limited to, povidone iodine, alcohols, benzalkonium chloride, benzethonium chloride, and parachlorometaxylenol (PCMX).

### Essential Fatty Acids

In particular embodiments, a composition of the invention includes one or more essential fatty acids, such as omega-3 (alpha-linolenic acid) and omega-6 (linoleic acid), which modulate inflammation and promote healing.

### Assessment of Wound Healing

Methods of characterizing wound healing activity are known in the art and are described herein. In one embodiment, a composition of the invention is assayed for an effect on cell migration using any conventional method known in the art. In one embodiment, cell migration is assayed using a two-dimensional in vitro wound system. In one embodiment of this assay, the cells are skin cells, such as fibroblasts, keratinocytes, or endothelial cells. Comparable migration assays are also useful in the methods of the invention and are well known to the skilled person and comprise, for example, the Boyden chamber method, the scratch assay, the colloidal gold assay and an assay based on the migration in a fibrin matrix. In a scratch assay, cells are seeded on a tissue culture plate and are grown to confluency. The confluent cell layer is then wounded under standard conditions with a plastic pipet tip to create a cell free zone. Subsequently, test substances can be added after and migration into the cell free zone can be monitored by photo documentation of identical locations in the scratch.

For the colloidal gold assay, coverslips are coated with colloidal gold salts and covered with a suitable substratum, for example Collagen I. Cells, for example keratinocytes or fibroblasts are plated on the cover slip and allowed to migrate for several hours. Afterwards the cells are fixed in formaldehyde and migration tracks can be analyzed using computer assisted image analysis. In the assay based on the migration in a fibrin matrix, cells are plated onto a fibrin matrix that is obtained from freeze-dried surgical fibrinogen and distributed onto culture dishes before clotting. The fibrin matrix is transparent and therefore suitable for microscopic analysis of the cells. Suitable cells, for example fibroblasts or keratinocytes are incubated on the matrix for 24 hours, fixed with formaldehyde and tunnels generated by migrating cells in the matrix are examined by light microscopy.

Compositions of the invention identified as useful in an in vitro assay may be tested, if desired, in an in vivo assay system carried out in mice, dogs, or horses, for example, to determine whether the application of a composition of invention to a wound alters the healing of the wound. This can be done, for example, by measuring the rate of re-epithelialization.

The compositions of this invention may be modified by appending appropriate functionalities to enhance selective biological properties. Such modifications are known in the art and include those which promote wound healing.

Compositions identified using the methods described herein are useful treating a chronic wound, or for a related disease and/or disorder or symptom thereof. Such methods comprise administering a therapeutically effective amount of a pharmaceutical composition comprising a compound of the formulae herein to a subject (e.g., a mammal such as a human). Thus, one embodiment is a method of treating a subject suffering from or susceptible to the formation of a chronic wound, or a related disease or disorder or symptom thereof. The method includes the step of administering to the mammal a therapeutic amount of an amount of a compound herein sufficient to treat the disease or disorder or symptom thereof, under conditions such that the disease or disorder is treated. Identifying a subject in need of such treatment can be in the judgment of a subject or a health care professional and can be subjective (e.g. opinion) or objective (e.g. measurable by a test or diagnostic method).

### Formulation of Pharmaceutical Compositions

Compositions of the invention are useful for promoting wound healing. In particular embodiments, a biopolymer (e.g., chitin, chitosan, collagen, cellulose, alginate, dextrose) and one or more natural healing agents (e.g., manuka honey) is dissolved in an acidic solvent (e.g., acetic, propionic, citric, lactic, hypochlorous, or phosphoric acid). Acidic solvents useful in compositions of the invention include, but are not limited to, acetic or citric acid in a concentration sufficient to dissolve one or more biopolymers provided in powder form in a liquid solution. The viscosity of the solution can be varied as desired by pouring a known mass of biopolymer into an acid solvent solution; and mixing until the powdered dissolves. Other agents may be added to solution. Depending on the viscosity of the solution, it may be helpful to apply negative pressure (e.g., a vacuum) to the resulting solution prior to filling a container of choice (bottle, tube, syringe, etc.) for delivery of the composition to a wound.

In particular embodiments, the resulting composition comprises nano-particles or micro-particles (e.g., silver, magnesium), which are useful to prevent bacterial, microbial, or fungal contamination of the solution itself or the treated local wound or wound environment. Further nano- or micro-particulate metals and non-metals have been shown to interact at the cellular level, which may have a preservative, or active pharmaceutical or medicinal effect.

Accordingly, the compositions of the present invention are formulated for topical administration. Suitable formulations include gels, sprays, washes, ointments, and creams. Administration of each compound of the combination may be by any suitable means that results in a concentration of the compound that, combined with the other compound, is effective. Each compound can be admixed with a suitable carrier substance, and is generally present in an amount of 1-95% by weight of the total weight of the composition. If desirable, the compounds can be formulated together. In particular embodiments, chitosan is present in a composition of the invention in the range of 0.00001 to 10 wt %, in the range of 0.00001 to 5 wt %, or in the range of 0.00001 to 1 wt %. In particular embodiments, chlorhexidine is present in the range of 0.00001 to 5 wt %, in the range of 0.00001 to 1 wt %, from 0.00001 to 0.5 wt %, or from 0.00001 to 0.25 wt %. In particular embodiments, the acid(s) solvent is present in the range of 0.00001 to 5 wt %, preferably from 0.00001 to 1 wt %. In particular embodiments, water makes up the balance of the solution, and represents no less than 90 wt % of the entire solution.

In particular embodiments, a composition of the invention is a hydrogel characterized by a final solution density between 0.96-1.04, a pH between 2.5-6.5, and a viscosity of 50 cps-500 cps. In particular embodiments, a composition of the invention is a paste having a pH between 2.5-6.5, and a viscosity of 500 cps-1000 cps.

In particular embodiments, a composition of the invention comprises a plant-based pomace. Pomace is the pulpy residue that remains after the plant materials have been pressed or crushed to extract its juice. Fruits, vegetables, herbs, and plants may be provided in the form of pomace, such as a powder, liquid, solid or concentrated form based on the whole or a part of the flora. Common examples of pomace include, but are not limited to, apple powder, beet powder, beetroot powder, banana or banana peel powder, and compositions from berries (for example, blueberry, blackberry, raspberry, strawberry, cranberry). In other embodiments, a composition of the invention comprises natural fruit, vegetable, or plant-based compositions, i.e. pomaces, powders, liquids, lyophilized components, partial, or whole, high in antioxidants, including but not limited to berries, apples, or citrus fruits. In one embodiment, the composition comprises pomaces, powders, liquids, lyophilized components, partial, or whole, having anti-inflammatory properties, including but not limited to beets, beetroot, other root-based flora, or bananas.

The pharmaceutical compositions may be formulated for topical use according to conventional pharmaceutical practice (see, e.g., Remington: The Science and Practice of Pharmacy, (20th ed.) ed. A. R. Gennaro, 2000, Lippencott Williams & Wilkens, Philadelphia, Pa., and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time period after administration, using controlled release topical formulations.

Therapeutic compositions suitable for topical application include conventional anhydrous or aqueous preparations including ointments, lotions, creams, pastes, jellies, sprays, aerosols, and oils. These preparations can include oleaginous, aqueous, or emulsion-type bases. Optionally, topically applied formulations can be covered with an occlusive or semi-occlusive dressing.

### Means for Delivery

Compositions of the invention are provided in any conventional manner useful for the application of therapeutics to a wound. Such means include sprays, ointments, hydrogels, dressings, plasters, compresses or other topical forms which contain compounds according to the invention. Thus, it is possible to administer agents comprising suitable additives or auxiliary substances, such as physiological sodium chloride solution, demineralized water, stabilizer, proteinase inhibitors, gel formulations, such as white vaseline, low-viscosity paraffin and/or yellow wax, etc., topically and locally in order to exert an immediate and direct effect on the wound healing process. The topical administration of therapeutic compositions can be effected, for example, in the form of a hydrogel, ointment, cream, a foam, an aerosol spray, an injection, a gel matrix or a sponge or in the form of drops or washings.

In other embodiments, an agent described herein is delivered to a wound using a polymeric material or blend of polymeric materials (e.g., chitosan, collagen, cellulose, etc.) to form a delivery system. Preferably, the polymer contains an effective amount of a composition of the invention (e.g., chlorhexidine, silver). This polymeric delivery system provides for the systematic and/or locally administration of a desired amount of a therapeutic agent.

Other embodiments of the present invention include wound-healing devices configured and produced as woven sheets. Such sheets provide a means for temporarily treating and sealing an open wound. Additionally, the compositions of the invention may be provided in combination with any other pharmacologically active agents that promote the healing of the tissue within and around the wound.

Compositions of the invention may be applied to a wound to promote healing. Such compositions are administered directly to an injured area, for example, by spraying, spreading, sprinkling, packing, implanting, inserting or applying or by any other administration means to open wounds on the body.

### Methods of Use

The invention is directed to methods of treating and preventing wounds in mammals (e.g., canine, feline, equine, human). The non-human young and adult animals for which the treatment methods are suitable may include different animal types, genera, or species. In general, young and adult farm animals, animals bred or kept for various purposes, such as sport (e.g., racing, riding, dressage), transport, domestic, companion (e.g., dogs, cats), industrial uses (e.g. hauling, pulling, plowing), and the like, are particularly amenable to treatment according to the methods of the invention. For example, encompassed by the methods of the invention is the treatment of adult or young non-human animals, such as camels (calves), sheep (lambs), rams, horses (foals), pigs (piglets), goats (kids), bison/buffalo (calves), llamas, donkeys, mules, yaks, etc. Neonatal, young and adult exotic animals, such as zoo animals of various species, are also embraced by the treatments of the invention. In preferred aspects, young and adult horses are animal subjects that are particularly amenable to the methods and compositions of the invention.

### Kits or Pharmaceutical Systems

The present compositions may be assembled into kits or pharmaceutical systems for use in ameliorating a wound or promoting wound healing. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the compounds of the invention. In various embodiments, such kits are labelled for use in wound treatment or include directions for the use of the compositions of the invention to promote wound healing.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Hydrogels

Hydrogels for use in wound healing were produced. The hydrogels were made according to the following Formulas.

### Formula 1. PRIME Wound Hydrogel

0.8% chitosan
0.5% acetic acid
0.2% chlorhexidine digluconate
98.5% water

### Formula 2. PRIME Wound Hydrogel

0.7% chitosan
1.0% acetic acid
0.2% chlorhexidine digluconate
98.1% water

### Formula 3. PRIME Wound Hydrogel

0.85% chitosan
1.0% acetic acid
0.2% chlorhexidine digluconate
97.95% water

### Formula 4. PRIME Wound Hydrogel

0.9% chitosan
0.5% acetic acid
0.2% chlorhexidine digluconate
98.4% water

### Formula 5. PRIME Wound Hydrogel

0.7% chitosan
0.5% acetic acid
0.2% chlorhexidine digluconate
98.6% water

### Example 2: Sprays

Sprays for use in wound healing were produced. The sprays were made according to the following Formulas.

### Formula 5: PRIME Wound and Skin Spray

0.25% chitosan
0.5% acetic acid
0.2% chlorhexidine digluconate
99.05% water

### Formula 6: PRIME Wound and Skin Spray

0.15% chitosan
1.0% acetic acid
0.2% chlorhexidine digluconate
98.65% water

### Formula 7. PRIME Wound and Skin Spray

0.2% chitosan
1.0% acetic acid
0.2% chlorhexidine digluconate
98.6% water

### Formula 8. PRIME Wound and Skin Spray

0.1% chitosan
0.5% acetic acid
0.2% chlorhexidine digluconate
99.2% water

### Example 3: Silver Wound Gel

Gels for use in wound healing were produced. The gels were made according to the following Formulas.

### Formula 9. PRIME Silver Wound Gel

0.8% chitosan
0.5% acetic acid
0.2% chlorhexidine digluconate
1% (or 100 ppm) silver nanoparticles
97.5% water

### Formula 10. PRIME Silver Wound Gel

0.8% chitosan
0.5% acetic acid
0.2% chlorhexidine digluconate
1.5% (or 150 ppm) silver nanoparticles
97.0% water

### Example 4: Ointment

Ointments for use in wound healing were produced. The ointments contained the following ingredients.

### Formula 11. PRIME Wound Ointment

4% chitosan
1% acetic acid
0.2% chlorhexidine digluconate
94.8% water

### Formula 12. PRIME Wound Ointment

4% chitosan
2% acetic acid
0.2% chlorhexidine digluconate
93.8% water

### Formula 13. PRIME Wound Ointment

2% chitosan
1% acetic acid
0.2% chlorhexidine digluconate
96.8% water

### Example 5: Chlorhexidine and Silver Inhibited MRSA

Infection control is the most vital tool in the treatment of chronic wounds in veterinary care. Methicillin-resistant Staphylococcus aureus (MRSA) is one of the primary causes of bacterial infections in animals. Antimicrobial resistance has become a major concern in veterinary medicine. Veterinary antimicrobial sprays and hydrogels are used in the treatment of wounds and prevention of bacterial infections. The following experiments were conducted to evaluate the antimicrobial activity of seven commercially available veterinary care sprays and hydrogels against MRSA.

Products listed in Table 1 (below) were acquired at pet product suppliers.

| Product | Active ingredients |
|---|---|
| Vetericyn Plus Spray | Hypochlorous acid |
| Theracyn | Hypochlorous acid |
| PRIME Wound and | Chlorhexidine |
| Skin Spray | |
| PRIME Wound | Chlorhexidine |
| hydrogel | |
| PRIME Silver Wound | Silver, Chlorhexidine |
| gel | |
| Remedy Recovery | Benzalkonium Chloride |
| Purishield | Cholan-24-oic acid, |
| | antimicrobial peptides |

MRSA solution was diluted by taking 500 μl of solution and suspending it in 16.25 ml of TSB to give 1 in 33 (1/33) concentration.

### ZOI Inhibition Assays

1. Sterile blank paper disks 6 mm in diameter were loaded with 30 μl of solution in eight different groups.
2. Petri dishes were inoculated with SCO1, a clinical isolate of MRSA, and loaded discs were placed on inoculated dishes.
   The diameter of the cleared area around each disk was measured using Image J analysis software, and the inhibitory activity of the solution was assessed from the measured diameter.

### Planktonic Growth Studies

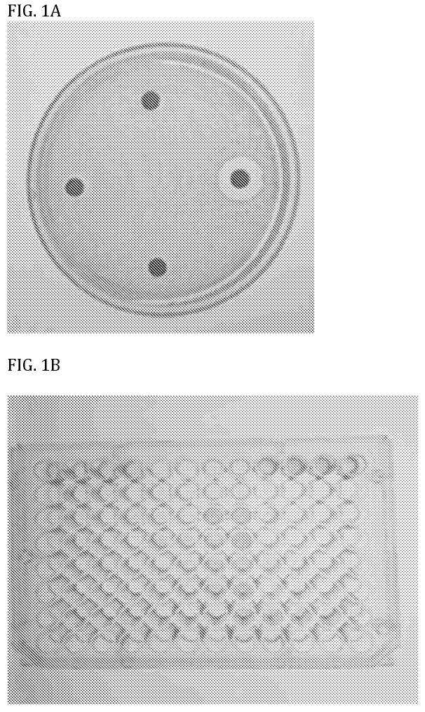

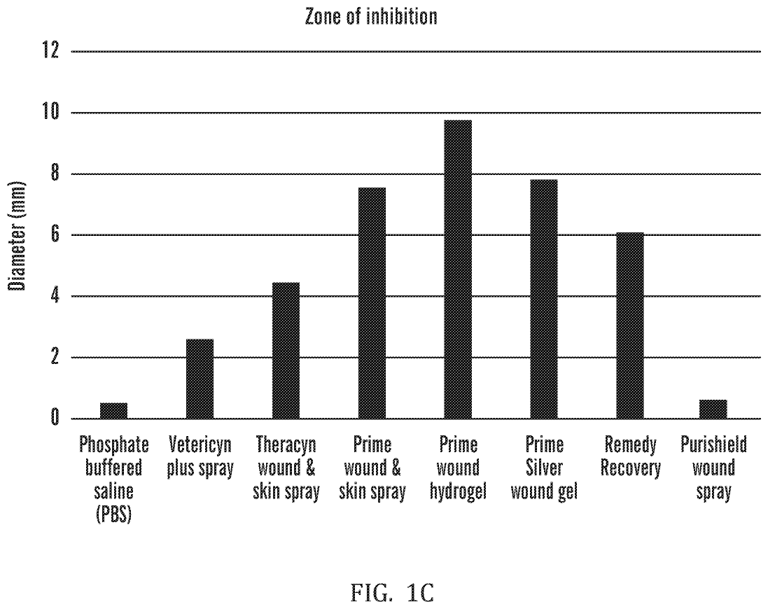

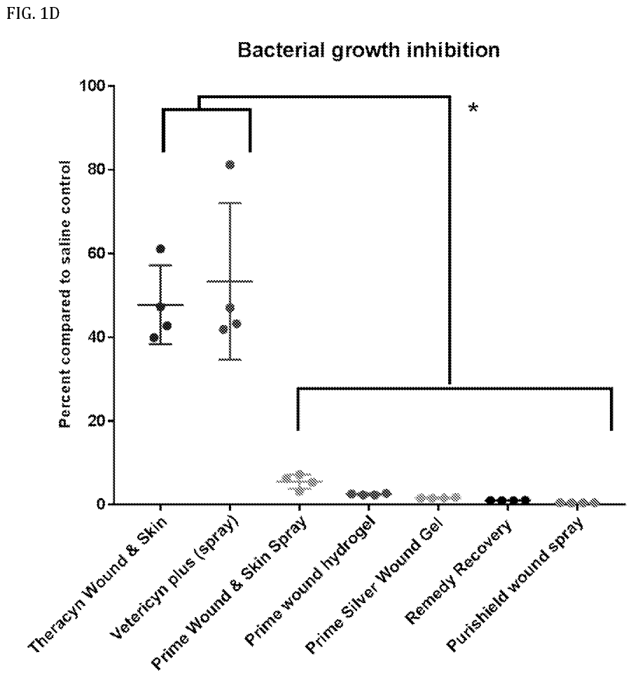

1. Wound care solutions in table 1 were added to tryptic soy broth in wells of a 96 well plate at a 1:4 dilution.
2. After 24 hours, turbidity was read and Bactieter glo (100 μL) added to the wells to assess ATP production by metabolically active MRSA cells.
   Results of the ZOI and Planktonic growth analysis are shown in FIGS. 1A, 1B, 1C and 1D. FIGS. 1B and 1C show exemplary plates analyzed. This analysis showed that a combination of chlorhexidine and silver was effective in inhibiting bacterial growth.

### Example 6: Biofilm Kill Assay

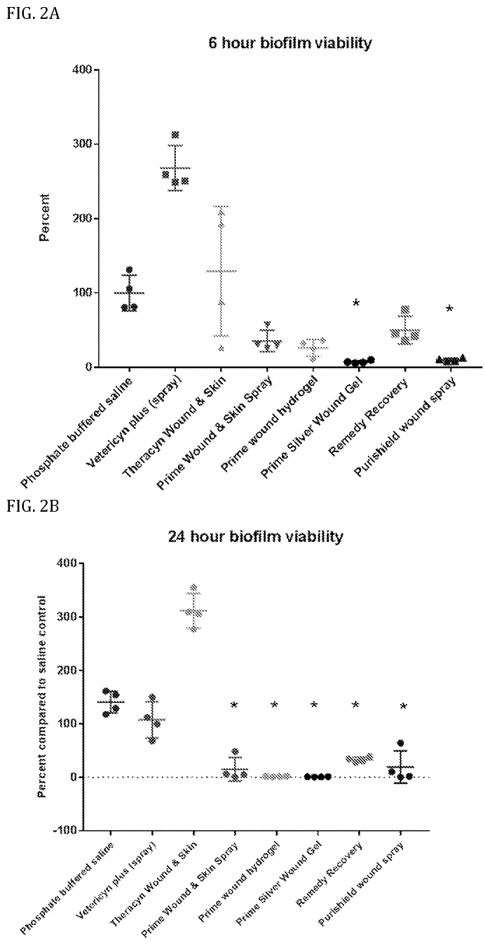

Biofilm was formed in 96 well plates. The solutions were added to the wells. The antimicrobial activity was measured after 6 and 24 hours of direct contact. Planktonic growth was removed and washed with phosphate buffered saline. One hundred μl of liquid sprays and saline controls were added to the wells. Results of biofilm assays are shown at FIGS. 2A and 2B.

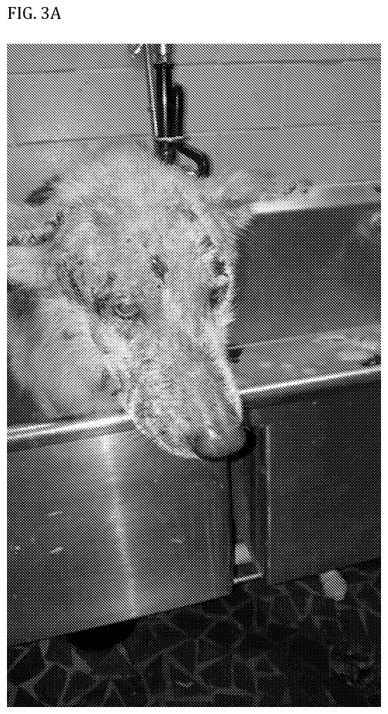

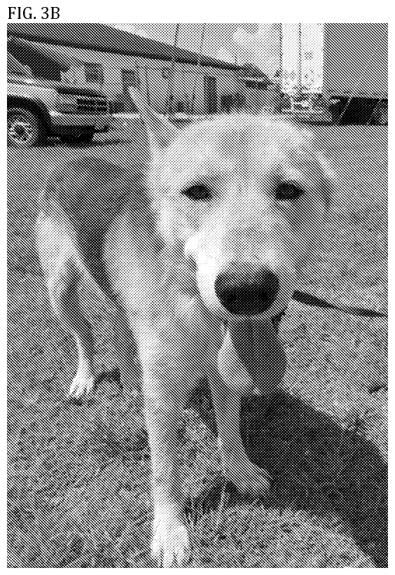

The products containing chlorhexidine and silver showed the largest zone of inhibition. Minimal inhibition was observed for products containing hypochlorous acid or antimicrobial peptide groups. Benzalkonium chloride showed enhanced inhibition in ZOI (FIG. 3). With bacterial growth inhibition assays and Bactieter glo we observed 99% antimicrobial inhibition for groups containing chlorhexidine, silver, benzalkonium chloride, and antimicrobial peptides (FIG. 2A). The 6 hour and 24 hours direct contact bactericidal assays showed significant activity for the products containing silver, chlorhexidine, and antimicrobial peptides (FIG. 2B). The products containing chlorhexidine and/or silver showed enhanced antimicrobial activity in all three-assays. Topical sprays for wounds prove more beneficial when infection is prevented with minimal cellular toxicity. Chitosan is effective for healing of chronic wounds in animals as it promotes healing while inducing minimal cellular toxicity. Gram positive bacteria were susceptible to benzalkonium chloride. These studies showed that greater contact time prevents the formation of biofilms. The products with chlorhexidine, chitosan, and silver have high efficiency for topical use for treatment of MRSA infections in equine practice.

Antimicrobial sprays and hydrogels are used for wound healing and inhibition of microbial growth. In this studies described herein, the antimicrobial activity of eight commercially available veterinary care sprays and hydrogels was compared against representative pathogenic Gram-positive and Gram-negative bacteria (Pseudomonas aeruginosa and Staphylococcus aureus). Microbial inhibition of each group was measured using zone inhibition (ZOI) testing), turbidity, and Bac TiterGlo™ viability assays. Results proved that products containing chitosan and chlorhexidine showed inhibitory action against both strains of bacteria in all three assays. Silver in combination with chitosan and chlorhexidine had more inhibition of Pseudomonas aeruginosa than all other groups. Products based on hypochlorous acid failed to show inhibition in ZOI, turbidity, and viability assays. The product with cholan-24-oic acid did not inhibit the microbial growth in ZOI, but did inhibit growth in turbidity and viability assays. This may be due to the detergent nature of this product, which could break open bacterial membranes in solution form. A benzalkonium chloride-based product showed inhibition in all studies. For wounds that have known contamination or infection, especially with Gram-negative microorganisms, different products might be recommended based on these results. Antimicrobial susceptibility testing will help veterinarians to choose appropriate antimicrobial therapy for wound healing.

### Example 7: In Vivo Wound Healing

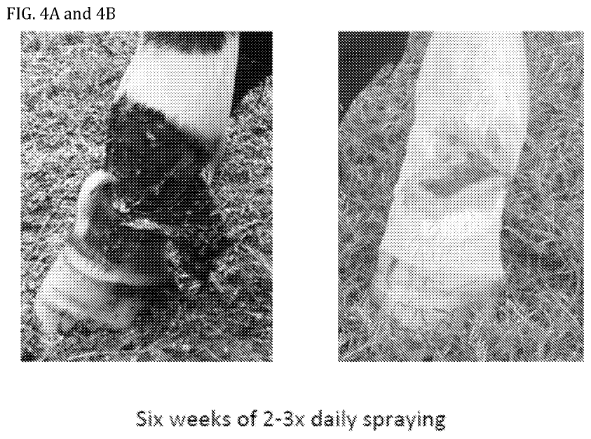

Compositions of the invention were used to treat canine demodectic mange (FIGS. 3A and 3B) as well as a variety of equine wounds (FIGS. 4A, 4B, 5A, 5B, 6A, 6B). The canine shown in FIG. 3A was treated for three weeks twice daily by spraying with a composition of the invention.

A horse with a degloved hoof injury was treated by spraying a composition of the invention 2-3 times daily for six weeks (FIGS. 4A and 4B).

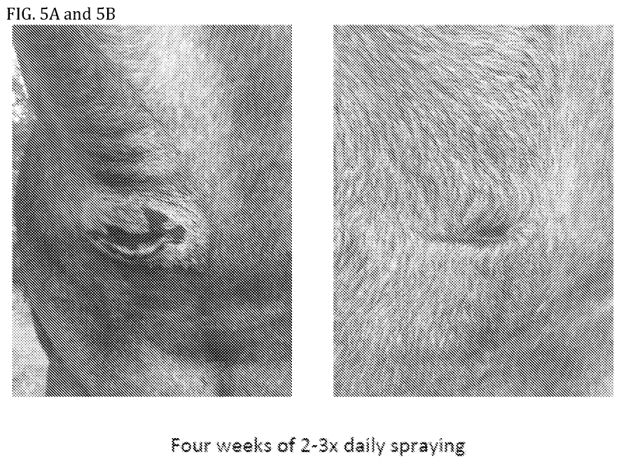

A horse with a puncture wound to the chest caused by running into a broken gate was treated by spraying a composition of the invention 2-3 times daily for four weeks (FIGS. 5A and 5B).

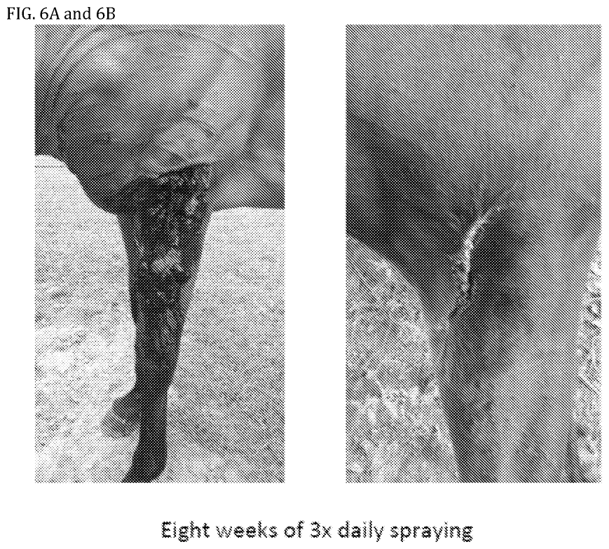

A horse with a foreleg wound caused by running into a barbed wire fence was treated by spraying a composition of the invention 3 times daily for eight weeks (FIGS. 6A and 6B).

The results described herein above, were carried out as follows. Commercially-available veterinary preparations for topical application with infection prevention claims (Table 1) were purchased at local supply stores.

Zone of Inhibition Evaluations

A modification of the Kirby-Bauer disk diffusion assay measures the ability of liquid and gel components to inhibit bacterial growth as well as the diffusion of active components into the surrounding medium. Sterile blank paper disks 6 millimeter (mm) in diameter (Becton, Dickinson and Company) were loaded with 30 microliters (μl) of solution in eight different groups. Petri dishes were inoculated with SCO1, a clinical isolate of MRSA. Paper disks were placed on inoculated dishes. Bactericidal and inhibitory activity of solutions were assessed by measuring the diameter of clearing seen surrounding each disk on the Petri dish using Image J analysis software.

Planktonic Growth Studies

Liquid sprays in each group were evaluated in static culture of MRSA. Briefly, wound care solutions in table 1 were added to Tryptic soy broth in wells of a 96-well plate at a 1:4 dilution. Because many of the solutions change the color of the solution or form precipitates, bacterial growth was assessed using BacTiter Glo (Promega). This luciferase-based bacterial viability assay lyses the cells and correlates the ATP released to the metabolic activity of viable cells. The solution was added to wells after overnight growth to assess ATP production by metabolically active cells. Luminescence values were compared to positive and negative controls with and without bacteria, respectively.

Biofilm Time-Kill Assay

The activity of solutions in direct contact with pre-formed biofilms was assessed after 6 and 24 hours of direct contact. SCO1 was inoculated in each well of a 96-well plate and allowed to grow and form biofilm overnight. Planktonic growth was removed and wells were washed gently with phosphate buffered saline. Liquid sprays and saline controls were pipetted at a volume of 100 μL at full strength.

Statistical Analysis

ANOVA with Holm-Sidak post hoc testing was used to determine significant differences between groups at the 5% significance level.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A composition comprising an effective amount of a biopolymer, an antiseptic, and an anti-inflammatory agent.

2. The composition of claim 1, further comprising an acid selected from the group consisting of acetic, propionic, citric, lactic, hypochlorous, and phosphoric acid; wherein the biopolymer is selected from the group consisting of chitosan, chitin, collagen, alginate, dextran, and combinations thereof

3. (canceled)

4. The composition of claim 1, further comprising a natural healing agent and/or a bittering agent in an amount sufficient to prevent licking or ingestion of the composition.

5. The composition of claim 4, wherein the natural healing agent is honey.

6. A composition comprising an effective amount of a biopolymer selected from the group consisting of chitosan, collagen, cellulose, alginate, and dextran; an antiseptic selected from the group consisting of chlorhexidine gluconate, povidone iodine, alcohol, benzalkonium chloride, benzethonium chloride, and parachlorometaxylenol (PCMX); and an acid that is acetic acid or citric acid.

7. A composition comprising an effective amount of chitosan, acetic acid, and chlorhexidine digluconate in an excipient for topical administration.

8. The composition of claim 1, further comprising silver nanoparticles.

9-10. (canceled)

11. An ointment, spray, gel, or hydrogel composition comprising an effective amount of chitosan, acetic acid, and chlorhexidine digluconate in an excipient for topical administration.

12. The composition of claim 1, further comprising silver nanoparticles.

13. The composition of claim 1, wherein the composition comprises between about 1-10% chitosan; composition comprises between about 1-3% acetic acid; comprises between about 0.01-3% chlorhexidine digluconate.

14-15. (canceled)

16. The composition of claim 1, wherein the composition comprises electrolized water containing dissolved sodium chloride; comprises hypochlorous acid or sodium hydroxide; comprises ketoconazolel; or comprises sodium hypochlorite (NaOCl).

17-21. (canceled)

22. The composition of claim 1, wherein the composition is characterized by a viscosity greater than 500 centipoise (cP).

23. The composition of claim 1, further comprising a fruit, vegetable, or plant-based pomace, powder or liquid derived from a berry, apple, citrus fruit, beet, root, or banana.

24. A method for inhibiting the proliferation of a Gram-negative or positive bacteria, the method comprising contacting the bacteria with a composition of claim 1.

25. The method of claim 24, wherein the bacteria is methicillin-resistant Staphylococcus aureus (MRSA) or Pseudomonas aeruginosa.

26. A method for treating a wound in a mammal the method comprising contacting the wound with a composition of claim 1.

27. The method of claim 26, wherein the mammal is a human, equine, canine, or feline.

28. The method of claim 26, wherein the method treats a topical, superficial or soft tissue infection.

29. The method of claim 26, wherein the method treats an umbilical cord or navel and prevents infection, bacterial contamination, or aids in umbilical drying out.

30. The method of claim 26, wherein the method treats or prevents itching, irritated skin, and hot spots.
